(19) United States
(12) Patent Application Publication
(10) Pub. No.: US 2024/0032837 A1
(43) Pub. Date: Feb. 1, 2024

(54) ADHD SEVERITY EVALUATION SYSTEM USING CORRELATIONS BETWEEN DATA GATHERED BY VR AND ADHD-RS

(71) Applicants: HIPPO T&C, INC., Suwon-si (KR); RESEARCH & BUSINESS FOUNDATION SUNGKYUNKWAN UNIVERSITY, Suwon-si (KR); SAMSUNG LIFE PUBLIC WELFARE FOUNDATION, Seoul (KR)

(72) Inventors: Tai Myoung CHUNG, Seoul (KR); Ha Min SON, Suwon-si (KR); Yoo Sook JOUNG, Seoul (KR)

(73) Assignees: HIPPO T&C, INC., Suwon-si (KR); RESEARCH & BUSINESS FOUNDATION SUNGKYUNKWAN UNIVERSITY, Suwon-si (KR); SAMSUNG LIFE PUBLIC WELFARE FOUNDATION, Seoul (KR)

(21) Appl. No.: 18/352,520

(22) Filed: Jul. 14, 2023

(30) Foreign Application Priority Data
Jul. 26, 2022 (KR) 10-2022-0092740

(51) Int. Cl. A61B 5/16; A61B 5/12

(52) U.S. Cl. CPC A61B 5/168; A61B 5/163; A61B 5/12

(57) ABSTRACT

An ADHD severity evaluation system using correlations between data gathered by VR and an ADHD-RS is provided. The ADHD severity evaluation system allows for gathering accurate data on a subject in an isolated and controlled environment by using VR, and improves the accuracy of ADHD diagnosis by measuring the severity and progression of ADHD. This can help determine an appropriate method and procedure for treating and/or relieving ADHD.

### CROSS-REFERENCE TO RELATED APPLICATIONS

This application claims the benefit of Korean Patent Application No. 10-2022-0092740, filed on Jul. 26, 2022, the contents of which are all hereby incorporated by reference herein in their entirety.

### TECHNICAL FIELD

The present disclosure relates to an ADHD severity evaluation system using correlations between data gathered by VR and an ADHD-RS, and more particularly, to a system for evaluating ADHD severity based on information on a subject that is gathered in a controlled and isolated environment.

This work was supported by the Institute of Information and communications Technology Planning and Evaluation (IITP) grant funded by the Korea government (MSIT) (no. 2020-0-00990, Platform Development and Proof of High Trust and Low Latency Processing for HeterogeneousAtypicalLarge Scaled Data in 5G-IoT Environment).

### BACKGROUND

Attention deficit/hyperactivity disorder (ADHD) is a disorder commonly found in children, characterized by constant inattention, distractibility, hyperactivity, and impulsivity. For the diagnosis of ADHD, the Diagnostic and Statistical Manual of Mental Disorders (DSM) are used which enables third persons such as mental health clinicians or parents to observe and evaluate ADHD in subjects.

Among the recent developments is a method of diagnosing ADHD by taking videos of behaviors a subject does and analyzing them. In relation to this, Korean Laid-Open Patent No. 10-2017-0094532 was disclosed.

However, the conventional art is problematic in that it does not ensure objectivity because the subject is not exposed to an isolated environment, and it does not measure the severity and progression of ADHD. Severity and progression assessment is very crucial in determining how to treat a subject with ADHD and what treatment procedure is needed. Also, an ADHD diagnosis system that ensures objectivity is required.

### SUMMARY

The present disclosure provides an ADHD severity evaluation system using correlations between data gathered by VR and an ADHD-RS, in order to solve the problems in the conventional art, such as not ensuring objectivity in the evaluation of ADHD symptoms and not being able to assessing severity and progression.

In an aspect of the present disclosure, there is provided a method an ADHD severity evaluation system using correlations between data gathered by VR and an ADHD-RS, the system including: a content storage part that stores content to which a subject is exposed in a VR environment; a VR implementation data storage part that stores VR implementation data gathered from the subject who is exposed to the content in the VR environment; an ADHD rating scale storage part containing an ADHD rating scale; and an artificial intelligence part that derives classification values according to the ADHD rating scale by receiving the VR implementation data, wherein the artificial intelligence part calculates scores for evaluation criteria set forth in the Diagnostic and Statistical Manual of Mental Disorders (DSM) based on the VR implementation data, and creates a first ADHD rating scale based on the scores for the evaluation criteria.

In an initial state, the artificial intelligence part may set a first weight of 1 between the VR implementation data and the evaluation criteria in the Diagnostic and Statistical Manual of Mental Disorders (DSM), set a second weight of 1 between the evaluation criteria and the first ADHD rating scale, and update the first weight and the second weight based on ADHD rating scale data inputted by an observer of the subject by observing the subject.

The artificial intelligence part may update the first weight and the second weight by increasing the first weight and the second weight if there is a high correlation between the associated data or decreasing the first weight and the second weight if there is a low correlation between the associated data.

The VR implementation data may include: movement data including at least one of eye movement data, head movement data, and hand movement data from the subject; voice data including at least one of response time, volume, and pitch variability which is extracted from the subject's voice; and activity data related to how instructions specified in the content are followed.

The VR implementation data storage part may include reference data gathered from normal children and children with ADHD who implement the VR content, wherein the reference data is classified and stored based on the child's gender and age.

The artificial intelligence part may load the VR implementation data of the subject, calculate each data value in the VR implementation data as a percentile for the reference data to store the percentile for each of the evaluation criteria, calculates a mean percentile for each of the evaluation criteria, classify the evaluation criteria into an inattention domain and a hyperactivity/impulsivity domain, calculate a mean percentile for the evaluation criteria corresponding to the inattention domain, and calculate a mean percentile for the evaluation criteria corresponding to the hyperactivity/impulsivity domain.

The artificial intelligence part may calculate scores for the criteria in the first ADHD rating scale by using a softmax function, based on the means calculated for the inattention domain and the hyperactivity/impulsivity domain.

The ADHD severity evaluation system using correlations between data gathered by VR and an ADHD-RS according to the present disclosure allows for gathering accurate data on a subject in an isolated and controlled environment by using VR, and improves the accuracy of ADHD diagnosis by measuring the severity and progression of ADHD. This can help determine an appropriate method and procedure for treating and/or relieving ADHD.

### BRIEF DESCRIPTION OF THE DRAWINGS

FIG. 1 is a block diagram of an ADHD severity evaluation system using correlations between data gathered by VR and an ADHD-RS according to the present disclosure.

FIG. 2 is a view illustrating a flow of data that is processed in the ADHD severity evaluation system using correlations between data gathered by VR and an ADHD-RS.

FIG. 3 is a table showing an example of evaluation criteria included in the DSM-5.

FIG. 4 is a table showing an example of evaluation criteria that match observation criteria.

FIG. 5 is a table showing an example of how data that matches each observation criterion for the subject is normalized and expressed as a percentile.

FIG. 6 is a table showing an example of a process for calculating a mean percentile for each evaluation criterion for the subject.

FIG. 7 is a table showing an example of a process for calculating the subject's severity as a percentile for an inattention domain and a hyperactivity/impulsivity domain.

FIG. 8 is a table showing a distribution of ADHD-RS scores for girls.

FIG. 9 is a table showing a distribution of ADHD-RS scores for boys.

FIG. 10 is a view illustrating an example of learning data inputted for the artificial intelligence part to learn.

FIG. 11 is a conceptual diagram showing initial settings for weights in the learning by the artificial intelligence part.

FIG. 12 is a conceptual diagram showing the concept of weight correction in the learning by the artificial intelligence part.

### DETAILED DESCRIPTION

Hereinafter, an ADHD severity evaluation system using correlations between data gathered by VR and an ADHD-RS according to an embodiment of the present disclosure will be described in detail with reference to the accompanying drawings. In the following description of the embodiments, different components may have different names in the art. However, if they are functionally similar and identical, they may be deemed as equivalent components even if a modified embodiment is employed. In addition, reference numerals added to different components are indicated for convenience of explanation. However, what is depicted in the drawings in which these reference numerals are indicated do not limit the components to the ranges these drawings cover. Likewise, even if an embodiment is employed in which some of the components in the drawing are modified, they can be deemed as equivalent components if they are functionally similar and identical. Further, if a component is construed as a component that should be included, from the level of a person having ordinary skill in the art, a description thereof will be omitted.

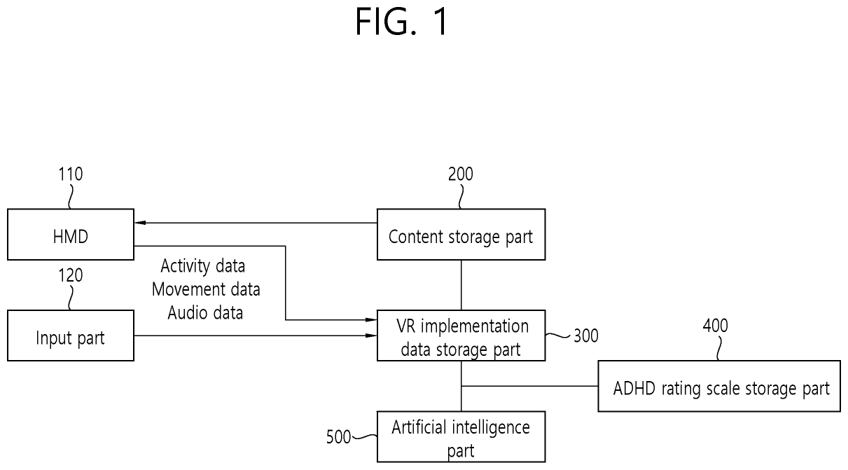

FIG. 1 is a block diagram of an ADHD severity evaluation system using correlations between data gathered by VR and an ADHD-RS according to the present disclosure.

Referring to FIG. 1, the ADHD severity evaluation system 1 using correlations between data gathered by VR and an ADHD-RS according to an embodiment of the present disclosure may include a head-mounted display (HMD) 110, an input part 120, a content storage part 200, a VR implementation data storage part 300, an ADHD rating scale storage part 400, and an artificial intelligence part 500.

The HMD 110 is configured such that a subject whose ADHD symptoms are to be evaluated can wear, and helps get the subject fully immersed and bring more objectivity to a situation where the subject is presented with. The HMD 1 may include a plurality of sensors and a display part. The plurality of sensors is configured to collect various information generated from the subject when the subject is exposed to content. The plurality of sensors is comprising a microphone, camera, accelerometer. The each sensor may be configured to recording the voice, track the wearer's eye movement and head movement and angle respectively.

The input part 120 may be configured to allow the subject to perform a particular input. The input part 120 may be configured to move a pointer or enter a particular command, in relation to the content. The input part 120 may be a stick with buttons, for example, which the subject is supposed to hold.

The content storage part 200 may store VR content. The VR content may be configured to expose the subject to a particular environment and provide particular tasks or games in order. The content storage part 200 may store content including video and audio data. For example, the VR content may be a game that involves a mission like “getting your school bag packed” or a game that requires concentration such as “moving a ball”.

In the case of the game of getting your school bag packed, for example, the subject is supposed to pack their school bag by reading a checklist of items they need to bring to school. In case of a game in which parts are assembled, content requiring progression according to an order or position among a plurality of parts may be included.

As another example, the ball moving game may include content that requires the subject to move a ball into a tube of the same color as the ball by using a shovel.

The VR implementation data storage part 300 is configured to store various information inputted by the subject and the HMD 1 the subject is wearing by means of the input part 120. Signals generated by the HMD 1 and the input part 120 may include activity data, movement data, and voice data.

The activity data may include interactions with content, for example, information on particular commands, command input times, numbers of command inputs, etc. related to different instructions. The movement data may include information on the posture, angle, and movement of the head and the direction of movement of the eyes, which is obtained by the HMD, and information on the posture, angle, and movement of the hands obtained by an acceleration sensor provided in the input part 120. The voice data may be obtained by utterances made by the subject, and may include at least one of response time, volume, and pitch variability.

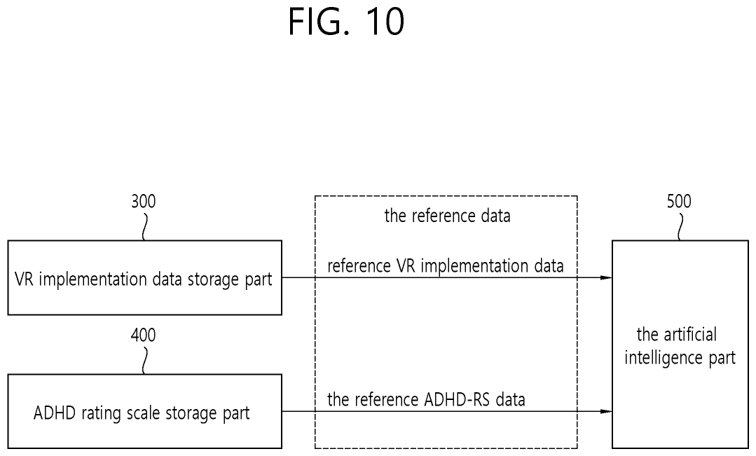

The ADHD rating scale storage part 400 may store data on the ADHD-RS. In this instance, the data may include information gathered by observing and keeping track of the subject, which may be utilized as reference data later on. Such reference data will be described later with reference to FIGS. 10 to 12.

Meanwhile, the content storage part 200, the VR implementation data storage part 300, the ADHD rating scale storage part 400 described above may be hardware that can function as a database, and may work in an integrated manner.

The content stored in the above-described content storage part 200 is not limited to the above-described games, but may store various content by updates.

Moreover, the VR implementation data storage part 300 may include data gathered from a diversity of subjects. The ADHD-RS storage part 400 also may store results of evaluation for a diversity of subjects. In this instance, information on the genders and ages of the diversity of subjects may be stored as well.

The artificial intelligence part 500 is configured to classify the evaluation results according to the ADHD rating scale based on input the VR implementation data.

The artificial intelligence part 500 extracts meaningful data from the VR implementation data, calculates percentiles for different evaluation criteria set forth in the Diagnostic and Statistical Manual of Mental Disorders (DSM), and produces results for the subject for each criterion on the ADHD rating scale based on the percentiles. In this case, the subject's percentile for each evaluation criterion may statistically show the level the subject is at in comparison to other children of the same age group.

The artificial intelligence part 500 may be configured to include a linear regression model, for example. Also, an analysis algorithm composed of differentiable functions may be configured as a convolutional neural network which excels at extracting features after gathering large amounts of data. Finally, the artificial intelligence part 500 calculates scores on the ADHD rating scale by using a softmax function.

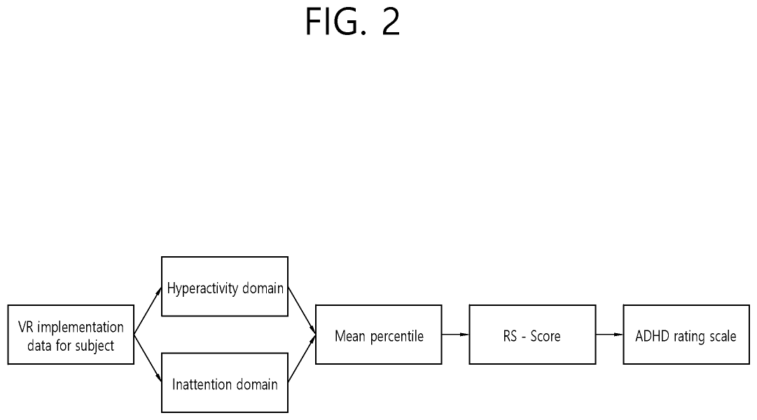

Hereinafter, data processing performed in the ADHD severity evaluation system using correlations between data gathered by VR and an ADHD-RS according to an embodiment of the present disclosure will be described with reference to FIG. 2.

FIG. 2 is a view illustrating a flow of data that is processed in the ADHD severity evaluation system using correlations between data gathered by VR and an ADHD-RS.

Referring to FIG. 2, numerical values of VR implementation data obtained in the present disclosure are calculated by artificial intelligence according to the evaluation criteria in the DSM, and mean percentiles for a hyperactivity/impulsivity domain and an inattention domain in the ADHD rating scale are calculated from the numerical values for the evaluation criteria. Afterwards, the calculated percentiles are converted to ADHD rating scale scores by using a clinical standard table. Finally, the ADHD rating scale scores may be put into the evaluation criteria on the ADHD-RS and outputted.

That is, the artificial intelligence part 500 allows the subject to be exposed to content, and, once VR implementation data is inputted, may calculate scores for different criteria on the ADHD rating scale based on the VR implementation data.

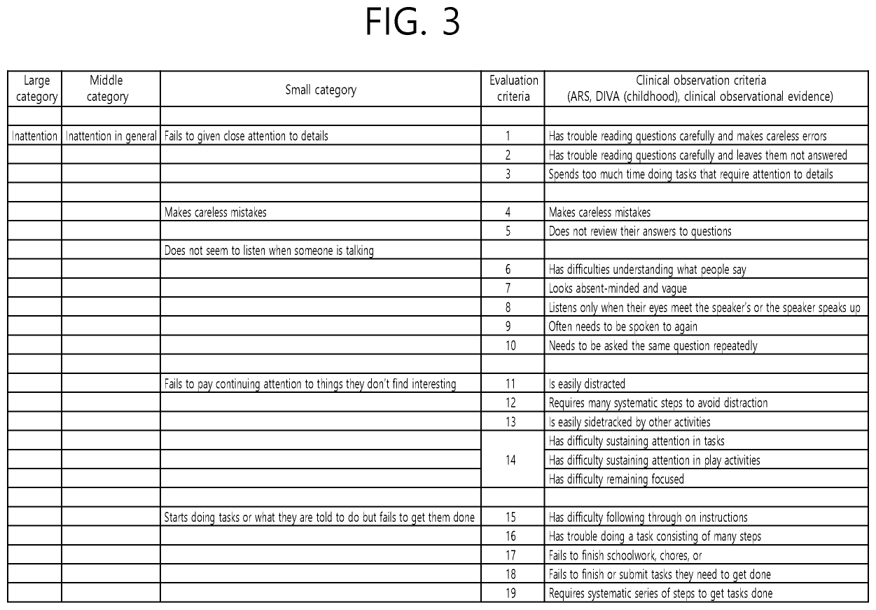

Hereinafter, data processed by the artificial intelligence part 500 will be described with reference to FIGS. 3 to 9.

FIG. 3 is a table showing an example of evaluation criteria included in the DSM-5.

Referring to FIG. 3, the Diagnostic and Statistical Manual of Mental Disorders (DSM-5) contains criteria for determining symptoms of mental disorders. Specific examples of clinical observation criteria include “Has trouble reading questions carefully and makes careless errors”, “Has trouble reading questions carefully and leaves them not answered”, “Spends too much time doing tasks that require attention to details”, “Makes careless mistakes”, “Does not review their answers to questions”, and so on. However, these clinical observation criteria are not quantified, and therefore the same subject may be evaluated differently if the subject is observed by different people.

On the other hand, these clinical observation criteria provide content like games and ensure objectivity through responses the subject makes when the subject is exposed to an isolated environment.

For example, when the subject is exposed to the ball moving game, if the subject moves the ball correctly in response to an instruction such as “put the yellow ball into the yellow tube”, an evaluation may be made with respect to the criterion “Has trouble reading questions carefully and makes careless errors”. Also, the evaluation results may differ depending on how often patterns in the subject's behavior are repeated with respect to this evaluation criterion. These results may be derived by integrating data obtained when the subject has finished through a certain type of content.

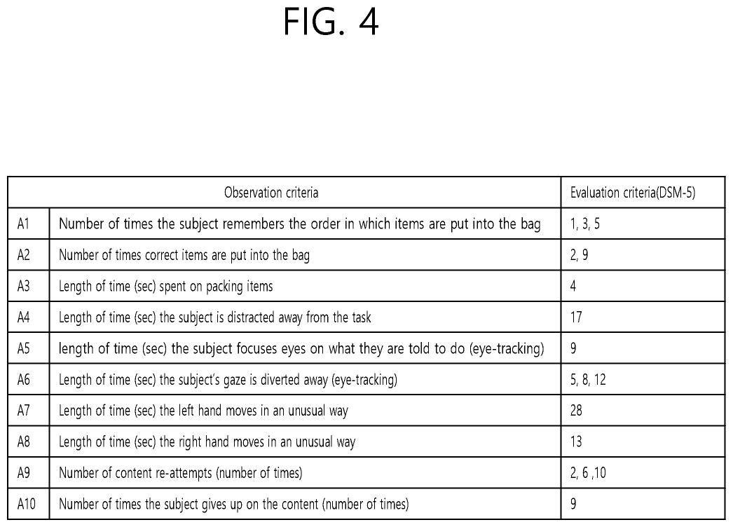

FIG. 4 is a table showing an example of evaluation criteria that match observation criteria.

FIG. 4 shows an example in which, in the case of VR content that involves the school bag packing game, meaningful observation criteria that can be extracted from VR implementation data match evaluation criteria set forth in the DSM-5. One observation criterion may match at least one evaluation criterion.

For example, Observation Criterion A1 “the number of times the subject remembers the order in which items necessary for school are put into the bag” may match the first to third evaluation criteria in the DSM-5: 1. Has trouble reading questions carefully and makes careless errors; 2. Has trouble reading questions carefully and leaves them not answered; and 3. Spends too much time doing tasks that require attention to details. In this instance, the number of times the subject remembers the order in which correct items are put into the bag may be stored in Evaluation Criteria 1, 2, and 3.

Meanwhile, the observation criteria may be set in various ways depending on the content, and values obtained from the VR implementation data may be stored in the evaluation criteria in the DSM-5 that match each observation criterion. Since one evaluation criterion may be associated with various observation criteria, a plurality of values may be stored. For example, Evaluation Criterion 2 (Has trouble reading questions carefully and leaves them not answered) may match Observation Criterion A2 (the number of times correct items are put into the bag) and Observation Criterion A9 (the number of content re-attempts).

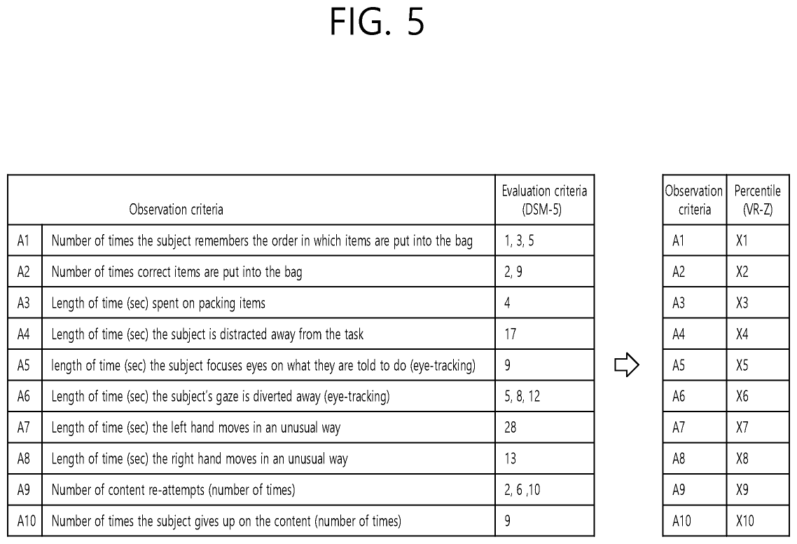

FIG. 5 is a table showing an example of how data that matches each observation criterion for the subject is normalized and expressed as a percentile.

The artificial intelligence part 500 statistically analyzes what level the subject is currently at with respect to the observation criteria A1, A2, . . . based on VR implementation data. In this case, the statistical analysis may be done based on data gathered from children of different ages and different genders apart from the current subject who have implemented VR for themselves. The percentile VR-Z for each observation criterion may be normalized and calculated by using the following Equation #1:

$$VR - Z ( n , n ) = \frac{RDt ( n , n ) - {μ}_{n , n}}{{σ}_{n , n}} ,$$

Where the RDt(n,n) is RDt(subject number, nth VR measured data) and μ is average of the VR measured data considering the child's gender and age. And σ is the standard deviation.

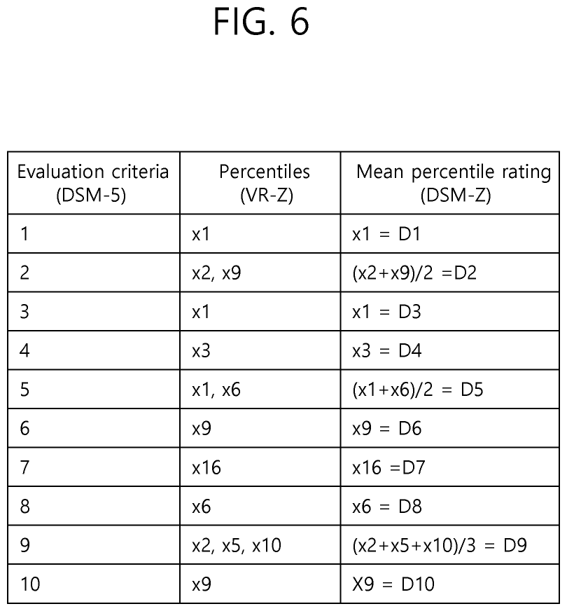

FIG. 6 is a table showing an example of a process for calculating a mean percentile for each evaluation criterion for the subject.

Referring to FIG. 6, each evaluation criterion may match at least one observation criterion as described above, and a processor calculates a mean percentile (DSM-Z) for the evaluation criterion based on each percentile (VR-Z). Calculation of the mean percentile (DSM-Z) may be made for all evaluation criterion.

For example, Evaluation Criterion 2 (Has trouble reading questions carefully and leaves them not answered) matches Observation Criterion A2 (the number of times correct items are put into the bag) and Observation Criterion A9 (the number of content re-attempts). In a statistical comparison to other children of the same age group, a percentile X2 for A2 (the number of times correct items are put into the bag) and a percentile X9 for A9 (the number of content re-attempts) may be derived from the VR implementation data of the current subject in shown FIG. 6.

And a mean percentile for each evaluation criterion may be calculated by using the following Equation #2:

$$DSM - Z ( x ) = {\sum }_{i = A}^{B} \frac{VRD ( i ) * w ( i )}{\vert DSM - Relation ( i ) \vert}$$

The weight (w(i)) is initially set to 1. accordingly, initial mean percentile D2 for Evaluation Criterion 2 may be equal to (X1+X2)/2. Through this procedure, mean percentiles D1, D2, . . . for every evaluation criterion may be calculated.

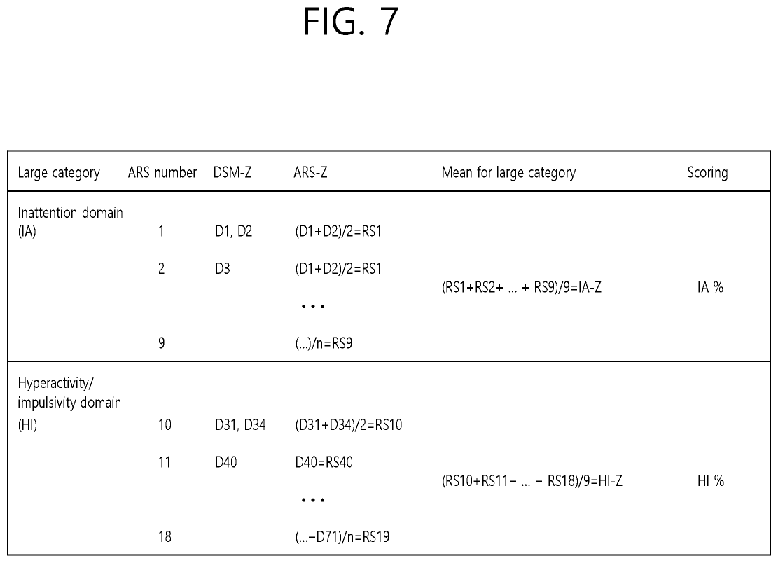

FIG. 7 is a table showing an example of a process for calculating the subject's severity as a percentile for an inattention domain and a hyperactivity/impulsivity domain.

Referring to FIG. 7, at least one evaluation criterion may be matched for each ARS number constituting the ADHD-RS of DSM-5. The ADHD-RS is broadly classified into two large categories: Inattention (IA) and Hyperactivity/Impulsivity (HI), and each large category has nine criteria.

Subsequently, at least one observation criterion may be matched at least one evaluation criterion in the DSM-5, and at least one evaluation criterion in the DSM-5 may be matched at least one criterion on the ADHD-RS.

In this instance, the processor is calculating the ARS-Z by averaging at least one the percentiles (D1, D2, . . . ) for at least one evaluation criterion that matches each ARS number on the ADHD-RS are calculated.

Afterwards, the mean percentile (IA-Z, HI-Z) for each large category may be calculated.

That is, the mean percentile for the inattention domain (IA-Z) may be calculated by the following Equation #3:

$$IA - Pcnt = {\sum }_{i = N}^{Z} \frac{VRD ( i ) * w ( i )}{\vert IA - Relation \vert} .$$

Likewise, the mean percentile for the hyperactivity/impulsivity domain (HI-Z) may be calculated by the following Equation #4:

$$HI - Pcnt = {\sum }_{i = A}^{M} \frac{VRD ( i ) * w ( i )}{\vert HI - Relation \vert} .$$

According to the above equations, the mean IA-Z of percentiles for the inattention domain is calculated based on each ARS-Z (RS1 , RS2, . . . , RS9) and weigh(i), and the mean HI-Z of percentiles for the hyperactivity/impulsivity domain is calculated based on ARS-Z(RS10, RS11, . . . , RS18) and weigh (i). Wherein the weight (i) is initially set to 1, but may change as the AI learns.

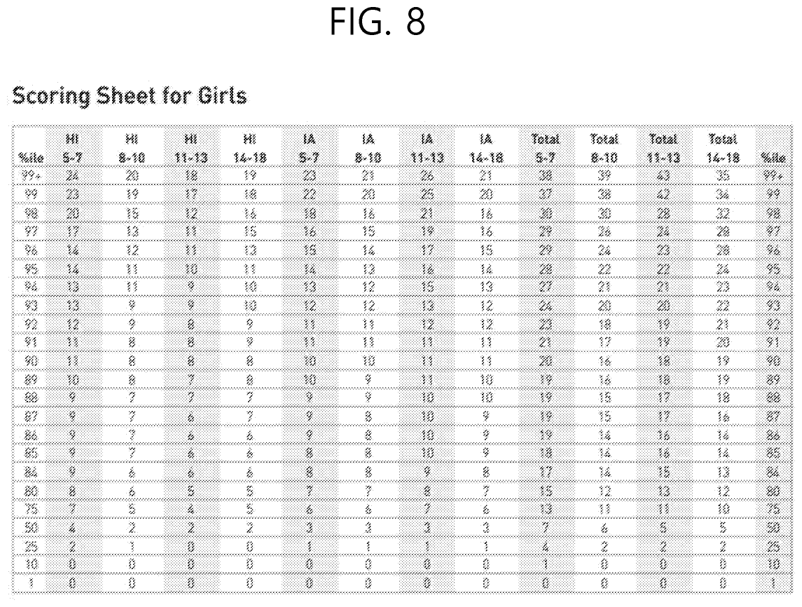

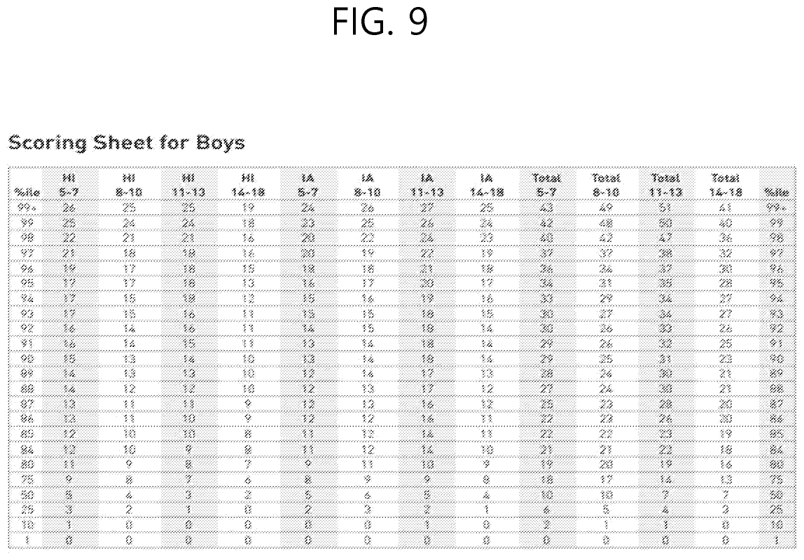

FIG. 8 is a table showing a distribution of ADHD-RS scores for girls, and FIG. 9 is a table showing a distribution of ADHD-RS scores for boys. FIGS. 8 and 9 show scores for different age groups in the HI domain and the IA domain, respectively. That is, these figures show percentile scores for different age groups from the highest 99th percentile to the lowest 1st percentile. For example, as shown in FIG. 8, if a 5-year-old girl is in the percentile rank for HI that corresponds to 0.5, that means that her HI score is 4, and if she is in the percentile rank for IA that corresponds to 0.8, that means that her IA score is 7.

The each score according to percentile scores are derived from the ADHD-RS

scoring sheet based on the mean percentile IA-Z for the inattention domain and the mean percentile HI-Z for the hyperactivity/impulsivity domain described with reference to FIG. 7.

Hereinafter, an example in which there are three VR data in relation to the above-described calculation of IA-Z and HI-Z will be described.

Assume that the data acquired by exposing the subject in the VR environment are, for example, [RDt(1,1)=5.4, RDt(1,2)=1.7, RDt(1,3)=2.6]. And in Equation #1, it is assumed that the weights for DSM #1, DSM #4, and DSM #7 among the evaluation criteria are 0.8, 1.0, 0.9 in the equation #1 by AI learning. Also, it is assumed that DSM #1, DSM #5, and DSM #7 match hyperactivity.

First, the Z-score (VR-Z) for each VR data point is calculated. Each VR-Z may be nomalized by subtracting the mean of data for other children of the same age group from RDt(n,n) and dividing the difference by a standard deviation. As an example, the calculated VR-Z value may be derived as VR-Z(1,1)=−0.51, VR-Z(1,2)=0, VR-Z(1,3)=−0.25. The calculated VR-Z may be converted into a percentile score (VR-D) by using a Z-table. Z-table is the values of the cumulative distribution function of the normal distribution. It is used to find the probability that a statistic is observed below, above, or between values on the standard normal distribution, and by extension, any normal distribution. Since probability tables cannot be printed for every normal distribution, as there are an infinite variety of normal distributions, it is common practice to convert a normal to a standard normal and then use the standard normal table to find probabilities. Then the percentile scores are: VR-D(1,1)=0.3, VR-D(1,2)=0.5, VR-D(1,3)=0.4 (If the time to complete one mission is fastest compared to peers, the percentage is 0%).

Next, DSM-Z is calculated (in this example, only DSM #1, DSM #5, and DSM #7 exist, and only one VR data matches each DSM, respectively). DSM-Z(1)=VR-D(1,1)/1=0.3, DSM-Z(5)=0.5, DSM-Z(7)=0.4 according to the equation #2.

Lastly, the mean percentile HI-Pnct for hyperactivity/impulsivity is calculated according to the equation #4 with DSM-Z and a weight: (0.3*0.8+0.51.0+0.4*0.9)/(0.8+1.0+0.9)=0.407. The corresponding percentile score can be found in FIG. 8 or FIG. 9. Percentage score that do not match the table may be scored by interpolation. At ages 5 to 7, the HI score was 5 in the top 50% and 2 in 25% (2^{nd} column, 2^{nd} and 3^{rd} rows from the bottom in FIG. 8). That is, if the subject is a 5-year-old girl, she may have a final score of 3.5 in the percentile rank corresponding to 0.407.

Afterwards, the artificial intelligence part 500 calculates a value for each ADHD-RS criterion by using a softmax function. It is assumed that, in the above example, the DSM criterion and the ADHD-RS criterion match as defined below, and both of them match hyperactivity/impulsivity: DSM-Z(1)=>ADHD-RS(2), DSM-Z(5)=>ADHD-RS(4), DSM(7)=>ADHD-RS(6). The softmax function is applied to the DSM-Z value to distributing the score of 3.5 calculated in the example. Here, the following calculation can be done by the softmax function: softmax([0.3, 0.5, 0.4])=[0.301, 0.332]. That is, ADHD-RS(2)=3.5*0.301=1.054, ADHD-RS(4)=3.5*0.367=1.285, ADHD-RS(6)=3.5*0.332=1.161.

The ADHD-RS provides four ratings: 0 indicates “Never or rarely”, 1 indicates “sometimes”, 2 indicates “often”, and 3 indicates “always or very often”. That is, if the subject is rated 1.054 for the ADHD-RS (2) “Fidgets with or taps hands or feet or squirms in seat”, rated 1.285 for the ADHD-RS (4) “Leaves seat in situations when remaining seated is expected”, and rated 1.161 for the ADHD-RS (6) “Runs about or climbs in situations where it is inappropriate”, it can be seen that the subject is rated “sometimes” for all of these three criteria. Another advantage is that decimal points can be used because of the benefits of Al analysis while the conventional ADHD-RS uses integers alone. For example, it can be construed that the subject is rated most poorly for the ADHD-RS (4) “Leaves seat in situations when remaining seated is expected, among the three criteria, for which the subject was given the highest score.

As a result, the subject's current scores for ADHD-RS criteria may be derived by artificial intelligence by using three VR data points.

Hereinafter, a learning process for the artificial intelligence part according to an embodiment of the present disclosure will be described with reference to FIGS. 10 to 12.

The term “reference data” described below means VR implementation data obtained when a diversity of children is exposed to VR content and data containing rating forms of the ADHD-RS the observer has filled out by observing each child. As used herein, the reference data as used herein may contain information on the gender and age of the child.

FIG. 10 is a view illustrating an example of learning data inputted for the artificial intelligence part to learn.

The learning by the artificial intelligence part is performed by loading reference VR implementation data from one child stored in the VR implementation data storage part and data from the ADHD-RS created by observing that child, and the learning is repeatedly performed as the reference data for each child is changed.

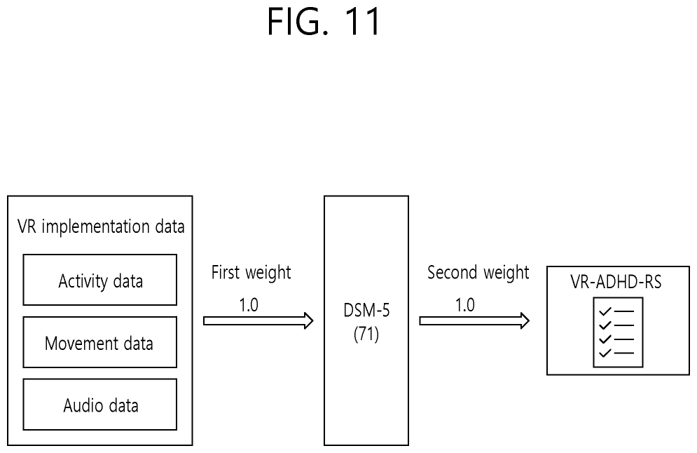

FIG. 11 is a conceptual diagram showing initial settings for weights in the learning by the artificial intelligence part.

Referring to FIG. 11, the artificial intelligence part sets a first weight between the observation criteria, which is information contained in the reference VR implementation data, and sets a second weight between the evaluation criteria in the DSM-5 and the criteria on the ADHD-RS. In this case, the first weight refers to a set of weights between nodes when the observation criteria are set as a plurality of nodes and the evaluation criteria in the DSM-5 are set as a plurality of nodes. Also, the second weight refers to a set of weights between the nodes for the DSM-5 and the nodes for the ADHD-RS when the criteria on the ADHD-RS are set as nodes. The artificial intelligence part starts learning after setting the first weight and the second weight to 1 in an initial state.

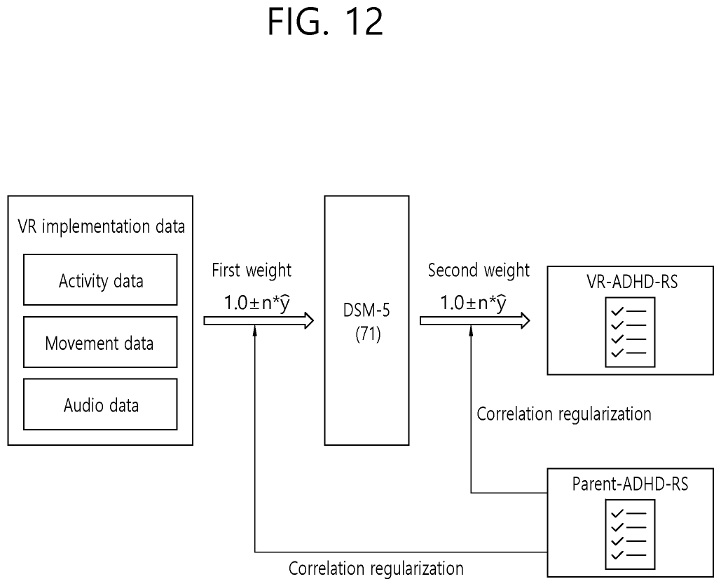

FIG. 12 is a conceptual diagram showing the concept of weight correction in the learning by the artificial intelligence part.

Referring to FIG. 12, the artificial intelligence part updates the weights by comparing values of VR ADHD-RS, which are classified and created from reference VR implementation data, and reference values of ADHD-RS which are created by the observer by actually observing the child. In this instance, the artificial intelligence part increases the first weight and the second weight for observation and evaluation criteria having a high correlation with each other, and decrease the first weight and the second weight for observation and evaluation criteria having a low correlation with each other. This is used as a method of regularization.

The above-described updating of the first and second weights is repeatedly performed as more data on the child is gathered, thereby increasing accuracy.

In relation to the learning by the artificial intelligence part, the problem of high cost and time for obtaining large amounts of data can be solved by constructing an intelligence-based system using the above-mentioned correlation regularization.

As described above, an ADHD severity evaluation system using correlations between data gathered by VR and an ADHD-RS according to the present disclosure allows for gathering accurate data on a subject in an isolated and controlled environment by using VR, and improves the accuracy of ADHD diagnosis by measuring the severity and progression of ADHD.

What is claimed is:

1. An ADHD severity evaluation system using correlations between data gathered by VR and an ADHD-RS, the system comprising:
a content storage part that stores content to which a subject is exposed in a VR environment;
a VR implementation data storage part that stores VR implementation data gathered from the subject who is exposed to the content in the VR environment;
an ADHD rating scale storage part containing an ADHD rating scale; and
an artificial intelligence part that derives classification values according to the ADHD rating scale by receiving the VR implementation data,
wherein the artificial intelligence part calculates scores for evaluation criteria set forth in the Diagnostic and Statistical Manual of Mental Disorders (DSM) based on the VR implementation data, and creates a first ADHD rating scale based on the scores for the evaluation criteria.

2. The ADHD severity evaluation system of claim 1, wherein, in an initial state, the artificial intelligence part sets a first weight of 1 between the VR implementation data and the evaluation criteria in the Diagnostic and Statistical Manual of Mental Disorders (DSM), sets a second weight of 1 between the evaluation criteria and the first ADHD rating scale, and updates the first weight and the second weight based on ADHD rating scale data inputted by an observer of the subject by observing the subject.

3. The ADHD severity evaluation system of claim 2, wherein the artificial intelligence part updates the first weight and the second weight by increasing the first weight and the second weight if there is a high correlation between the associated data or decreasing the first weight and the second weight if there is a low correlation between the associated data.

4. The ADHD severity evaluation system of claim 3, wherein the VR implementation data includes:
movement data including at least one of eye movement data, head movement data, and hand movement data from the subject;
voice data including at least one of response time, volume, and pitch variability which is extracted from the subject's voice; and
activity data related to how instructions specified in the content are followed.

5. The ADHD severity evaluation system of claim 3, wherein the VR implementation data storage part includes reference data gathered from normal children and children with ADHD who implement the VR content,
wherein the reference data is classified and stored based on the child's gender and age.

6. The ADHD severity evaluation system of claim 5, wherein the artificial intelligence part loads the VR implementation data of the subject, calculates each data value in the VR implementation data as a percentile for the reference data to store the percentile for each of the evaluation criteria, calculates a mean percentile for each of the evaluation criteria, classifies the evaluation criteria into an inattention domain and a hyperactivity/impulsivity domain, calculates a mean percentile for the evaluation criteria corresponding to the inattention domain, and calculates a mean percentile for the evaluation criteria corresponding to the hyperactivity/impulsivity domain.

7. The ADHD severity evaluation system of claim 6, wherein the artificial intelligence part calculates scores for the criteria in the first ADHD rating scale by using a softmax function, based on the means calculated for the inattention domain and the hyperactivity/impulsivity domain.
